(19) United States
(12) Patent Application Publication
(10) Pub. No.: US 2026/0077024 A1
(43) Pub. Date: Mar. 19, 2026

(54) AGENT FOR IMPROVING INTESTINAL FLORA

(71) Applicants: AMANO ENZYME INC., Nagoya-shi, Aichi (JP); HIROSHIMA UNIVERSITY, Higashihiroshima-shi, Hiroshima (JP); KEIO UNIVERSITY, Minato-ku, Tokyo (JP)

(72) Inventors: Manabu KURODA, Kakamigahara-shi, Gifu (JP); Shotaro YAMAGUCHI, Kakamigahara-shi, Gifu (JP); Norihisa KATO, Higashihiroshima-shi, Hiroshima (JP); Thanutchaporn KUMRUNGSEE, Higashihiroshima-shi, Hiroshima (JP); Yongshou YANG, Higashihiroshima-shi, Hiroshima (JP); Shinji FUKUDA, Tsuruoka-shi, Yamagata (JP)

(21) Appl. No.: 18/579,230

(22) PCT Filed: Jun. 20, 2022
(86) PCT No.: PCT/JP2022/024561

§ 371 Date: Jan. 12, 2024

(30) Foreign Application Priority Data
Jul. 14, 2021 (JP) 2021-116710

(51) Int. Cl. A61K 38/46; A61K 38/47; A61P 1/14

(52) U.S. Cl. CPC A61K 38/465; A61K 38/47; A61P 1/14; C12Y 301/01003; C12Y 302/01004

(57) ABSTRACT

An object of the present invention is to provide an agent for improving intestinal flora that can increase the numbers of bacteria of genus Bifidobacterium and bacteria of genus Lactobacillus in the intestine to improve the intestinal flora. (i) A lipase derived from a koji mold and/or (ii) a cellulase can increase the numbers of both bacteria of genus Bifidobacterium and bacteria of genus Lactobacillus in the intestine to exert an excellent effect of improving the intestinal flora.

### TECHNICAL FIELD

The present invention relates to an agent for improving intestinal flora that can improve the intestinal flora. More specifically, the present invention relates to an agent for improving intestinal flora that can increase the numbers of both bacteria of genus Bifidobacterium and bacteria of genus Lactobacillus in the intestine.

### REFERENCE TO SEQUENCE LISTING

A Sequence Listing submitted as an ASCII .txt file via Patent Center is hereby incorporated by reference in accordance with 35 U.S.C. § 1.52(e). The name of the .txt file for the Sequence Listing is 61922279_1.txt, the date of creation of the text file is Aug. 6, 2025, and the size of the text file is 1,289 bytes.

### BACKGROUND ART

In recent years, causal relationships between the intestinal environment and various diseases have been intensively investigated to reveal that improvement of the intestinal environment is effective in preventing or ameliorating various diseases. In the intestine, beneficial bacteria, such as bacteria of genus Bifidobacterium and bacteria of genus Lactobacillus, and bad bacteria, such as Escherichia coli, are present in a mixed manner. Forming a beneficial bacteria-dominated flora is important to form a healthy intestinal environment.

Probiotics, prebiotics, and the like have been developed in the past, and various materials by which beneficial bacteria become predominant to improve the intestinal flora have been proposed. It has also been reported that administration of enzymes can improve the intestinal flora.

For example, Non-Patent Literature 1 has reported that the intestinal flora can be improved in mice administered a lipase derived from Candida rugose. However, Non-Patent Literature 1 does not disclose that the lipase can increase the numbers of bacteria of genus Bifidobacterium and bacteria of genus Lactobacillus in the intestine. Thus, no lipases have been found that can increase the numbers of both bacteria of genus Bifidobacterium and bacteria of genus Lactobacillus in the intestine.

Non-Patent Literature 2 has also reported that in a pig fed with a specific enzyme blend consisting of an amylase, a protease, and a xylanase together with animal feed, the number of lactobacilli is increased and the number of Escherichia coli is decreased in the large intestine. Non-Patent Literature 3 has also reported that in a pig fed with a specific enzyme blend (Nopcozyme II; Diasham Resources Pte Ltd.) consisting of an amylase from Bacillus amyloliquefaciens, a protease from Bacillus subtilis, and a xylanase from Trichoderma together with animal feed, the number of lactobacilli is increased and the numbers of Salmonella and Escherichia coli are decreased. However, it has not been reported that celluloses have the effect of improving the intestinal flora.

### CITATION LIST

### Non-Patent Literature

Non-Patent Literature 1: Menden et al., Biomedicine & Pharmacotherapy, 2020, 130: 110579
Non-Patent Literature 2: Yi et al., Asian Astralas. J. Anim. Sci., 2013, 26: 1181-1188
Non-Patent Literature 3: Zhang et al., J. Amin. Sci., 2014, 92: 2063-2069

### SUMMARY OF INVENTION

### Technical Problem

In recent years, in response to the increasing interest in health promotion, the development of new materials by which beneficial bacteria such as bacteria of genus Bifidobacterium and bacteria of genus Lactobacillus become predominant to improve the intestinal flora has been desired.

It is thus an object of the present invention to provide an agent for improving intestinal flora that can increase the numbers of bacteria of genus Bifidobacterium and bacteria of genus Lactobacillus in the intestine to improve the intestinal flora.

### Solution to Problem

The present inventors have made extensive research to solve the foregoing problem and found that (i) a lipase derived from a koji mold and (ii) a cellulase can increase the numbers of both bacteria of genus Bifidobacterium and bacteria of genus Lactobacillus in the intestine to exert an excellent effect of improving the intestinal flora. The present inventors have made further research based on the findings, leading to the completion of the invention.

Thus, the present invention includes the following aspects.
Item 1. An agent for improving intestinal flora comprising (i) a lipase derived from a koji mold and/or (ii) a cellulase, as an active ingredient.
Item 2. The agent for improving intestinal flora according to Item 1, wherein the active ingredient is a lipase derived from Aspergillus niger.
Item 3. The agent for improving intestinal flora according to Item 1, wherein the active ingredient is a cellulase derived from a koji mold.
Item 4. The agent for improving intestinal flora according to Item 3, wherein the cellulase derived from a koji mold is a cellulase from Aspergillus niger.
Item 5. The agent for improving intestinal flora according to any one of Items 1 to 4, wherein the agent is used for increasing the numbers of bacteria of genus Bifidobacterium and bacteria of genus Lactobacillus in the intestine.
Item 6. A pharmaceutical preparation for oral administration for improving intestinal flora, comprising the agent for improving intestinal flora according to any one of Items 1 to 5.
Item 7. A food additive for improving intestinal flora, comprising the agent for improving intestinal flora according to any one of Items 1 to 5.
Item 8. A food or drink for improving intestinal flora, comprising the agent for improving intestinal flora according to any one of Items 1 to 5.
Item 9. A method for improving intestinal flora comprising orally applying (i) a lipase derived from a koji mold and/or (ii) a cellulase to an individual in need of improvement of the intestinal flora.
Item 10. A lipase derived from a koji mold and/or (ii) a cellulase for use in treatment to improve intestinal flora.
Item 11. Use of a lipase derived from a koji mold and/or (ii) a cellulase for the manufacture of an agent for improving intestinal flora.

### Advantageous Effects of Invention

The present invention uses (i) a lipase derived from a koji mold and/or (ii) a cellulase, which can increase the numbers of bacteria of genus Bifidobacterium and bacteria of genus Lactobacillus in the intestine to improve the intestinal flora, and thus is effective in, for example, forming a healthy intestinal environment, maintaining a healthy intestinal environment, and preventing or treating a disease and/or a symptom due in part to an unhealthy intestinal environment.

### DESCRIPTION OF EMBODIMENTS

An agent for improving intestinal flora of the present invention comprises (i) a lipase derived from a koji mold and/or (ii) a cellulase, as an active ingredient. The agent for improving intestinal flora of the present invention is described in detail below.

### [Active Ingredient]

In the agent for improving intestinal flora of the present invention, (i) a lipase derived from a koji mold and/or (ii) a cellulase is used as an active ingredient. By selecting and using (i) the lipase from a koji mold and/or (ii) the cellulase, the numbers of both bacteria of genus Bifidobacterium and bacteria of genus Lactobacillus in the intestine can be increased to improve the intestinal flora effectively.

In the agent for improving intestinal flora of the present invention, microorganisms from which the lipase is derived are not specifically limited as long as they are koji molds, and include, for example, Aspergillus niger, Aspergillus oryzae, Aspergillus sojae, Aspergillus saitoi, Aspergillus awamori, and Aspergillus flavus. Of these, a lipase derived from Aspergillus niger is suitably used in the present invention, because of its outstanding effect of increasing the numbers of bacteria of genus Bifidobacterium and bacteria of genus Lactobacillus in the intestine.

In the agent for improving intestinal flora of the present invention, microorganisms from which the cellulase is derived are not specifically limited and include, for example, microorganisms belonging to genus Aspergillus, such as Aspergillus niger, Aspergillus oryzae, Aspergillus sojae, Aspergillus saitoi, Aspergillus awamori, and Aspergillus flavus; microorganisms belonging to genus Trichoderma, such as Trichoderma reesei; and microorganisms belonging to genus Acremonium, such as Acremonium cellulolyticus. Of these, a cellulase derived from a microorganism of genus Aspergillus, particularly Aspergillus niger, is suitably used in the present invention, because of its outstanding effect of increasing the numbers of bacteria of genus Bifidobacterium and bacteria of genus Lactobacillus in the intestine.

Furthermore, in the agent for improving intestinal flora of the present invention, the type of cellulase used is not specifically limited and may be, for example, an endoglucanase, an exoglucanase, or a mixture thereof. In view of achieving a superior effect of improving the intestinal flora, the cellulase used in the present invention is preferably an enzyme preparation containing both an endoglucanase and an exoglucanase.

(i) the lipase derived from a koji mold and/or (ii) the cellulase used in the present invention may be in a purified form or a partially purified form containing other enzymes and the like.

While (i) the lipase derived from a koji mold and/or (ii) the cellulase can be produced using a known method, such as by microbial culture, these enzymes are commercially available and such commercial products may be used. Examples of commercially available lipases from koji molds include trade names “Lipase AP6” and “Lipase AP12” (lipase preparations derived from Aspergillus niger; Amano Enzyme Inc.); trade name “Sumizyme NLS” (a lipase preparation derived from Aspergillus niger; Shin Nihon Chemicals Corporation); and trade name Panamore (a lipase preparation derived from Aspergillus niger; DSM Japan K.K.). Examples of commercially available cellulases include trade name “Cellulase A ‘Amano’ 3” (a cellulase preparation derived from Aspergillus niger; Amano Enzyme Inc.); trade name “GODO Cellulase F” (a cellulase preparation derived from Trichoderma reesei; Godo Shusei Co., LTD.); trade name “BakeZyme X?CEL” (a cellulase preparation derived from Trichoderma reesei; DSM Japan K.K.); trade name “Sumizyme AC” (a cellulase preparation derived from Aspergillus niger; Shin Nihon Chemicals Corporation); and trade name “Celluzyme C” (a cellulase preparation derived from Trichoderma sp.; Nagase ChemteX Corporation). Of these commercial products, “Lipase AP12” and “Cellulase A ‘Amano’ 3” have an outstanding effect of increasing the numbers of bacteria of genus Bifidobacterium and bacteria of genus Lactobacillus in the intestine, and thus are suitably used in the present invention.

In the agent for improving intestinal flora of the present invention, one enzyme selected from (i) the lipase derived from koji molds and (ii) the cellulase may be used alone, or two or more enzymes from (i) the lipase derived from koji molds and (ii) the cellulase may be used in combination.

### [Dose]

The dose of the agent for improving intestinal flora of the present invention may be determined appropriately according to, for example, the type of active ingredient used, the type of product in which the agent is used, the use, the expected effect, and the form of application.

For example, when a lipase derived from a koji mold is used, the daily dose for an adult human as an amount of the lipase derived from a koji mold ingested or administered may be 1,000 to 100,000 U, preferably 3,000 to 60,000 U, and more preferably 5,000 to 30,000 U. As used herein, the lipase activity (U) is a value determined using the following method:

### (Method of Measuring Lipase Activity)

200 to 300 mL of a mixture of a 2 w/v % aqueous polyvinyl alcohol solution and olive oil at a volume ratio of 3:1 is placed in an emulsifier container and emulsified at 12,000 to 16,000 revolutions per minute for 10 minutes while cooling to 10° C. or less. After emulsification, the emulsion is left standing in a cool place for 1 hour, and, after confirming that the oil layer does not separate, the emulsion is used as a substrate solution.

First, 5 mL of the substrate solution and 4 mL of 0.1 mol/L phosphate buffer (pH 6.0) are precisely weighed into a flat-bottom test tube (30×120 mm), shaken, and left standing at 37±0.5° C. for 10 minutes; then, precisely 1 mL of a sample solution containing the sample whose activity is to be measured is added and immediately shaken. This solution is left standing at 37±0.5° C. for precisely 20 minutes, and then 10 mL of a 1:1 volume mixture of ethanol and acetone is added and shaken. Then, precisely 10 mL of a 0.05 mol/L aqueous sodium hydroxide solution is added, and additionally 10 mL of a 1:1 volume mixture of ethanol and acetone is added and shaken, and then excess sodium hydroxide is titrated with 0.05 mol/L hydrochloric acid (b mL) (indicator: 2 to 3 drops of phenolphthalein reagent). Separately, 5 mL of the substrate solution and 4 mL of 0.1 mol/L phosphate buffer (pH 6.0) are each precisely weighed into a flat-bottom test tube (30×120 mm), shaken, and left standing at 37±0.5° C. for 10 minutes; then, 10 mL of a 1:1 volume mixture of ethanol and acetone is added, and then precisely 1 mL of a sample solution containing the sample whose activity is to be measured is added and shaken. Then, precisely 10 mL of a 0.05 mol/L aqueous sodium hydroxide solution is added and thereafter, the same procedure is repeated, and titration is performed (a mL).

For titration, a pH meter is used, and 0.05 mol/L hydrochloric acid is added until the pH reaches 10.00. Under these conditions, the amount of enzyme that causes an increase in fatty acids by 1 micromole (μmol) per minute is defined as 1 U. The following equation is used for the calculation.

$$Lipase activity per g or mL of sample ( U / g or U / mL ) = 50 \times ( a - b ) \times 1 / 20 \times f \times 1 / M \qquad [ Expression 1 ]$$

a: titer value (mL) of blank
b: titer value (mL) of reaction solution
50: equivalent amount of fatty acids (micromole) per mL of 0.05 mol/L hydrochloric acid
20: reaction time (min)
M: amount of sample (g or mL) in 1 mL of test solution
f: factor of 0.05 mol/L hydrochloric acid

For example, when a cellulase is used, the daily dose for an adult human as an amount of the cellulase ingested or administered may be 2,000 to 200,000 U, preferably 6,000 to 120,000 U, and more preferably 10,000 to 60,000 U. As used herein, the cellulase activity (U) is a value determined using the following method:

### (Method of Measuring Cellulase Activity)

First, 4 mL of a 0.5 w/v % aqueous carmellose sodium solution (pH 4.5) is weighed into a 50 mL Nessler tube and left standing at 40±0.5° C. for 10 to 15 minutes, and then 1 mL of a sample solution containing the sample whose activity is to be measured is added and immediately shaken.

This solution is left standing at 40±0.5° C. for precisely 30 minutes, then 2 mL of the Somogyi reagent (a solution containing 2.4 w/v % anhydrous sodium carbonate, 1.2 w/v % sodium potassium tartrate tetrahydrate, 0.4 w/v % copper(II) sulfate pentahydrate, 1.6 w/v % sodium hydrogen carbonate, and 1.8 w/v % anhydrous sodium sulfate) is added and shaken, the tube is stoppered and heated in a boiling water bath for precisely 20 minutes and then cooled immediately. After cooling, 1 mL of the Nelson reagent is added and shaken well until complete dissolution of a red precipitate of cuprous oxide, and the solution is left standing at room temperature for about 20 minutes, adjusted to a total volume of 25 mL with 17 mL of water, and shaken well.

Absorbance (A30) at a wavelength of 500 nm is measured for the resulting solution, using water as a control. Separately, 2 mL of the Somogyi reagent is added to 4 mL of a 0.5 w/v % aqueous carmellose sodium solution (pH 4.5) and shaken, and then 1 mL of a sample solution containing the sample whose activity is to be measured is added and thereafter, the same procedure is repeated for the resulting solution, and absorbance (A0) is measured.

Also, separately, the same procedure is repeated using a 20 to 60 μg/mL glucose solution, and a glucose standard curve is prepared.

Under these conditions, the amount of enzyme that produces reducing sugars equivalent to 1 mg of glucose per minute is defined as 100 U. The following equation is used for the calculation.

$$Cellulase activity per g or mL of sample ( U / g or U / mL ) = G \times 1 / 30 \times 100 \times n \qquad [ Expression 2 ]$$

G: amount (mg) of produced glucose determined from glucose standard curve, using value (A30-A0)
1/30: conversion factor into per minute
100: conversion factor into 100 units
n: dilution factor per g or mL of sample

### [Use]

The agent for improving intestinal flora of the present invention can increase the number of beneficial bacteria such as bifidobacteria and lactic acid bacteria in the intestine, by the effect of (i) the lipase derived from a koji mold and/or (ii) the cellulase, to form a beneficial bacteria-dominated flora, and thus is used for the purpose of forming a healthy intestinal environment, maintaining a healthy intestinal environment, and the like. In particular, the agent for improving intestinal flora of the present invention has an excellent effect of increasing the numbers of both bacteria of genus Bifidobacterium and bacteria of genus Lactobacillus in the intestine, and thus can also be used as an agent for increasing the numbers of bacteria of genus Bifidobacterium and bacteria of genus Lactobacillus in the intestine.

Additionally, the agent for improving intestinal flora of the present invention can form a beneficial bacteria-dominated flora to form a healthy intestinal environment, and thus can also be used for the purpose of preventing or treating a disease and/or a symptom due in part to an unhealthy intestinal environment. Examples of the disease and/or symptom include decreased immunity, colorectal cancer, allergic disease, nonalcoholic steatohepatitis, obesity, and inflammatory bowel disease.

### [Form of Use]

The agent for improving intestinal flora of the present invention is orally applied by oral ingestion or oral administration. The agent for improving intestinal flora of the present invention is orally ingested or orally administered to exert the effect of improving intestinal flora after arriving at the intestine, and thus can be blended for use with various products such as foods and drinks, pharmaceutical preparations for oral administration, animal feed, and pet foods.

When the agent for improving intestinal flora of the present invention is blended with the various products, the products may contain probiotics and/or prebiotics as required together with the agent for improving intestinal flora of the present invention.

Examples of microorganisms used as the probiotics include lactic acid bacteria, bifidobacteria, and Bacillus subtilis var natto. Specific examples of the lactic acid bacteria include lactic acid bacteria of genus Lactobacillus, such as Lactobacillus casei, Lactobacillus acidophilus, and Lactobacillus plantarum; lactic acid bacteria of genus Enterococcus, such as Enterococcus faecalis, Enterococcus faecium, and Enterococcus hirae; and lactic acid bacteria of genus Streptococcus, such as Streptococcus bovis and Streptococcus thermophilus. Specific examples of the bifidobacteria include Bifidobacterium adolescentis, Bifidobacterium longum, Bifidobacterium bifidum, Bifidobacterium pseudolongum, and Bifidobacterium thermophilum. These probiotics may be used alone or in combination.

Examples of the prebiotics include xylooligosaccharides, fructooligosaccharides, soybean oligosaccharides, isomaltooligosaccharides, lacto-fructo-oligosaccharides, galactooligosaccharides, and lactulose. These prebiotics may be used alone or in combination.

The dosage form of a product with which the agent for improving intestinal flora of the present invention is blended may be any of a solid form, a semi-solid form, a liquid form, and the like, and is selected appropriately according to the type and use of the product.

When the agent for improving intestinal flora of the present invention is used for foods and drinks, (i) the lipase derived from a koji mold and/or (ii) the cellulase is provided as a food or a drink that exerts the effect of improving intestinal flora, either as is or in combination with other food materials or additive ingredients to be prepared in a desired form. Examples of the food and drink include foods for specified health uses, nutritional supplements, functional foods, and foods for patients, as well as common foods and drinks. Forms of these foods and drinks include, but are not limited to, supplements, such as tablets, granules, powders, capsules, and soft capsules; and drinks, such as energy drinks, fruit drinks, carbonated beverages, and lactic acid drinks.

When the agent for improving intestinal flora of the present invention is used for foods and drinks, the amount of the agent blended into the food and drink may be determined appropriately according to, for example, the type of active ingredient used, and the form of the food and drink, within a range satisfying the above-described dose.

For example, when the lipase derived from a koji mold is used as the agent for improving intestinal flora of the present invention and is provided in the form of a supplement, the amount of the lipase derived from a koji mold blended into the supplement may be 300 to 50,000 U/g, preferably 1,000 to 30,000 U/g, and more preferably 1,500 to 15,000 U/g.

For example, when the lipase derived from a koji mold is used as the agent for improving intestinal flora of the present invention and is provided in the form of a drink, the amount of the lipase derived from a koji mold blended into the drink may be 3 to 500 U/mL, preferably 10 to 300 U/mL, and more preferably 15 to 150 U/mL.

For example, when the cellulase is used as the agent for improving intestinal flora of the present invention and is provided in the form of a supplement, the amount of the cellulase blended into the supplement may be 600 to 100,000 U/g, preferably 2,000 to 60,000 U/g, and more preferably 3,000 to 30,000 U/g.

For example, when the cellulase is used as the agent for improving intestinal flora of the present invention and is provided in the form of a drink, the amount of the cellulase blended into the drink may be 6 to 1,000 U/mL, preferably 20 to 600 U/mL, and more preferably 30 to 300 U/mL.

When the agent for improving intestinal flora of the present invention is used in the field of foods and drinks, (i) the lipase derived from a koji mold and/or (ii) the cellulase can be provided as a food additive for improving intestinal flora, either alone or in combination with other ingredients.

When the agent for improving intestinal flora of the present invention is used for a pharmaceutical preparation for oral administration, the agent for improving intestinal flora of the present invention is provided as a pharmaceutical preparation for oral administration that exert the effect of improving intestinal flora, either alone or in combination with, for example, other pharmacologically active ingredients, pharmaceutically acceptable bases, or additives to be prepared in a desired form. Forms of the pharmaceutical preparation specifically include, but are not limited to, preparations for oral administration, such as tablets, granules, powders, capsules, soft capsules, syrups, and liquids.

Examples of the bases and additives used for manufacturing the pharmaceutical preparation for oral administration include aqueous bases such as water and alcohols, oily bases, excipients, binders, filling agents, disintegrants, lubricants, algefacients, pH-adjusting agents, thickeners, antioxidants, sequestering agents, surfactants, emulsifiers, solubilizers, solubilizing agents, flavoring agents, and antiseptics.

When the agent for improving intestinal flora of the present invention is used as a pharmaceutical preparation for oral administration, the proportion of the agent blended into the pharmaceutical preparation for oral administration may be determined appropriately according to, for example, the type of active ingredient used and the form of the pharmaceutical preparation for oral administration, within a range satisfying the above-described dose.

For example, when the lipase derived from a koji mold is used as the agent for improving intestinal flora of the present invention and is provided in the form of a solid or semi-solid pharmaceutical preparation for oral administration, the amount of the lipase derived from a koji mold blended into the solid or semi-solid pharmaceutical preparation for oral administration may be 300 to 50,000 U/g, preferably 1,000 to 30,000 U/g, and more preferably 1,500 to 15,000 U/g.

For example, when the lipase derived from a koji mold is used as the agent for improving intestinal flora of the present invention and is provided in the form of a liquid pharmaceutical preparation for oral administration, the amount of the lipase derived from a koji mold blended into the liquid pharmaceutical preparation for oral administration may be 3 to 500 U/mL, preferably 10 to 300 U/mL, and more preferably 15 to 150 U/mL.

For example, when the cellulase is used as the agent for improving intestinal flora of the present invention and is provided in the form of a solid or semi-solid pharmaceutical preparation for oral administration, the amount of the cellulase blended into the solid or semi-solid pharmaceutical preparation for oral administration may be 600 to 100,000 U/g, preferably 2,000 to 60,000 U/g, and more preferably 3,000 to 30,000 U/g.

For example, when the cellulase is used as the agent for improving intestinal flora of the present invention and is provided in the form of a liquid pharmaceutical preparation for oral administration, the amount of the cellulase blended into the liquid pharmaceutical preparation for oral administration may be 6 to 1,000 U/mL, preferably 20 to 600 U/mL, and more preferably 30 to 300 U/mL.

When the agent for improving intestinal flora of the present invention is used for animal feed or a pet food, the agent for improving intestinal flora of the present invention is provided as animal feed or a pet food that exerts the effect of improving intestinal flora, either alone or in combination with other animal feed ingredients to be prepared in a desired form. Examples of the animal feed ingredients used for the animal feed or pet food include cereal crops, such as corn, wheat, barley, and rye; bran, such as wheat bran and rice bran; dregs, such as corn gluten meal and corn germ meal; animal-derived feed, such as skimmed milk powder, whey, fish flour, and powdered bone; yeasts, such as brewer's yeast; calcium, such as calcium phosphate and calcium carbonate; vitamins; amino acids; and saccharides.

When the agent for improving intestinal flora of the present invention is used as animal feed or a pet food, the proportion of the agent blended into the animal feed or pet food may be determined appropriately according to, for example, the form and type of the animal feed or pet food, and the type of the animal to which the agent is applied.

For example, when the lipase derived from a koji mold is used, the amount of the lipase derived from a koji mold blended into the animal feed or pet food may be 2 to 200 U/g, preferably 6 to 120 U/g, and more preferably 10 to 60 U/g.

For example, when the cellulase is used, the amount of the cellulase blended into the animal feed or pet food may be 4 to 400 U/g, preferably 12 to 240 U/g, and more preferably 20 to 120 U/g.

### EXAMPLES

The present invention is described more specifically below with reference to examples, although the present invention should not be construed to be limited to these examples.

### Test Example 1: Influence of Lipase Derived from Aspergillus niger on Intestinal Flora

Using SD rats (male, 4 weeks old, from Hiroshima Institute for Experimental Animals), influence of the ingestion of Lipase AP12 (Amano Enzyme Inc.) (lipase activity: 13.5 U/mg), which is a lipase preparation from Aspergillus niger, on the intestinal flora was investigated. In the experiment, the rats were divided into two groups of a control group and a working group, with eight rats per group. The rats of each group were fed ad libitum for 14 days with the animal feed shown in Table 1.

TABLE 1

| | Control | Example 1 |
|---|---|---|
| Lipase AP12 | — | 0.1 |
| Beef tallow | 30.0 | 30.0 |
| Casein | 20.0 | 20.0 |
| L-Cystine | 0.3 | 0.3 |
| Vitamin Mix* | 1.0 | 1.0 |
| Mineral Mix* | 3.5 | 3.5 |
| Cellulose | 5.0 | 5.0 |
| Sucrose | 20.0 | 20.0 |
| Corn starch | 20.2 | 20.1 |
| Total | 100.0 | 100.0 |

The content of each ingredient in the table is in “% by weight”.
*refers to AIN-93 (a standard purified diet composition for mice and rats formulated by the American Institute of Nutrition (AIN)).

After 14 days of feeding with the animal feed, the body weights and the weights of cecal contents of the rats in each group were determined, and, after extraction of DNA from the cecal contents, quantitative PCR analysis (Yang et al. Biomed Rep 2015, 3: 715-720) was performed to quantify the copy numbers (numbers of bacteria) of bacteria of genus Bifidobacterium (Bifidobacterium spp.) and bacteria of genus Lactobacillus (Lactobacillus spp.) in the cecal contents. In this PCR analysis, the bacterial species-specific primers shown in Table 2 were used. Additionally, 16S rRNA gene sequencing analysis (Mishima et al. Am J Physiol Renal Physiol, 2018, 315: F824-F833) was performed to quantify the numbers of bacteria of genus Bifidobacterium and bacteria of genus Lactobacillus in the cecal contents.

TABLE 2

| | Nucleotide sequence of the primer used | Seq ID No. |
|---|---|---|
| For detection | Forward: CGCGTCYGGTGTGAAG | 1 |
| of | Reverse: CCCCACATCCAGCATCCA | 2 |
| Bifidobacterium | | |
| spp. | | |
| For detection | Forward: GAGGCAGCAGTAGGGAATCTTC | 3 |
| of | Reverse: GGCCAGTTACTACCTCTATCCTTCTTC | 4 |
| Lactobacillus | | |
| spp. | | |

The results are shown in Tables 3 and 4. In Example 1, the numbers of bacteria of genus Bifidobacterium and bacteria of genus Lactobacillus in the intestine were both higher than those in the control. That is, it was revealed that ingestion of the lipase derived from Aspergillus niger promoted the proliferation of bacteria of genus Bifidobacterium and bacteria of genus Lactobacillus in the intestine, leading to improvement of the intestinal flora.

TABLE 3
Average body weight, weight of cecal contents
and results of quantitative PCR analysis

| | | Control | Example 1 |
|---|---|---|---|
| Average body weight (g) | | 211 ± 3 | 213 ± 3 |
| Weight (g) of cecal contents | | 1.44 ± 0.06 | 4.72 ± 0.39* |
| Bacteria of genus | Copy number | 2.4 ± 0.7 | 62.8 ± 12.5* |
| Bifidobacterium | (×10⁸/g contents) | | |
| | Copy number | 3.5 ± 1.1 | 278.1 ± 44.5* |
| | (×10⁸/total contents) | | |
| Bacteria of genus | Copy number | 2.77 ± 0.78 | 7.56 ± 2.36 |
| Lactobacillus | (×10⁸/g contents) | | |
| | Copy number | 3.8 ± 1.08 | 38.2 ± 13.8* |
| | (×10⁸/total contents) | | |

The measured results in the table are expressed as mean ± standard error (SE), and
*indicates p < 0.05 by Student's t test.

TABLE 4
Results of 16S rRNA sequencing analysis

| | Control | Example 1 |
|---|---|---|
| Bacteria of genus Bifidobacterium | 0.34 ± 0.11 | 40.76 ± 4.89* |
| (% of total bacteria) | | |
| Bacteria of genus Lactobacillus | 6.98 ± 1.16 | 24.00 ± 6.82* |
| (% of total bacteria) | | |

The measured results in the table are expressed as mean ± standard error (SE), and
*indicates p < 0.05 by Student's t test.

### Test Example 2: Influence of Cellulase Derived from Aspergillus niger on Intestinal Flora

Using SD rats (male, 4 weeks old, from Hiroshima Institute for Experimental Animals), influence of the ingestion of Cellulase A “Amano” 3 (Amano Enzyme Inc.) (cellulase activity: 33 U/mg), which is a cellulase preparation derived from Aspergillus niger (a cellulase preparation exhibiting both endoglucanase activity and exoglucanase activity), on the intestinal flora was investigated. In the experiment, the rats were divided into two groups of a control group and a working group, with six to eight rats per group. The rats of each group were fed ad libitum for 14 days with the animal feed shown in Table 5.

TABLE 5

| | Control | Example 2 |
|---|---|---|
| Cellulase A “Amano” 3 | — | 0.1 |
| Beef tallow | 30.0 | 30.0 |
| Casein | 20.0 | 20.0 |
| L-Cystine | 0.3 | 0.3 |
| Vitamin Mix* | 1.0 | 1.0 |
| Mineral Mix* | 3.5 | 3.5 |
| Cellulose | 5.0 | 5.0 |
| Sucrose | 20.0 | 20.0 |
| Corn starch | 20.2 | 20.1 |
| Total | 100.0 | 100.0 |

The content of each ingredient in the table is in “% by weight”.
*refers to AIN-93 (a standard purified diet composition for mice and rats formulated by the American Institute of Nutrition (AIN)).

After 14 days of feeding with the animal feed, the body weights and the weights of cecal contents of the rats in each group were determined, and quantitative PCR analysis and 16S rRNA gene sequencing analysis were performed using the same methods as in Test Example 1 above to quantify the numbers of bacteria of genus Bifidobacterium and bacteria of genus Lactobacillus in the cecal contents.

The results are shown in Tables 6 and 7. In Example 2, the numbers of bacteria of genus Bifidobacterium and bacteria of genus Lactobacillus in the intestine were both higher than those in the control. That is, it was confirmed that ingestion of the cellulase derived from Aspergillus niger promoted the proliferation of bacteria of genus Bifidobacterium and bacteria of genus Lactobacillus in the intestine, leading to improvement of the intestinal flora.

TABLE 6
Average body weight, weight of cecal contents
and results of quantitative PCR analysis

| | | Control | Example 2 |
|---|---|---|---|
| Average body weight (g) | | 211 ± 3 | 214 ± 2 |
| Weight (g) of cecal contents | | 1.44 ± 0.06 | 5.05 ± 0.31* |
| Bacteria of genus | Copy number | 2.4 ± 0.7 | 5.59 ± 1.59 |
| Bifidobacterium | (×10⁸/g contents) | | |
| | Copy number | 3.5 ± 1.1 | 28.5 ± 9.2* |
| | (×10⁸/total contents) | | |
| Bacteria of genus | Copy number | 2.77 ± 0.78 | 13.92 ± 4.44* |
| Lactobacillus | (×10⁸/g contents) | | |
| | Copy number | 3.8 ± 1.08 | 78.1 ± 34.5* |
| | (×10⁸/total contents) | | |

The measured results in the table are expressed as mean ± standard error (SE), and
*indicates p < 0.05 by Student's t test.

TABLE 7
Results of 16S rRNA analysis

| | Control | Example 2 |
|---|---|---|
| Bacteria of genus Bifidobacteriuni | 0.34 ± 0.11 | 9.65 ± 2.12* |
| (% of total bacteria) | | |
| Bacteria of genus Lactobacillus | 6.98 ± 1.16 | 31.86 ± 8.91* |
| (% of total bacteria) | | |

The measured results in the table are expressed as mean ± standard error (SE), and
*indicates p < 0.05 by Student's t test.

1. An agent for improving intestinal flora comprising (i) a lipase derived from a koji mold and/or (ii) a cellulase, as an active ingredient.

2. The agent for improving intestinal flora according to claim 1, wherein the active ingredient is a lipase derived from Aspergillus niger.

3. The agent for improving intestinal flora according to claim 1, wherein the active ingredient is a cellulase derived from a koji mold.

4. The agent for improving intestinal flora according to claim 3, wherein the cellulase derived from a koji mold is a cellulase derived from Aspergillus niger.

5. The agent for improving intestinal flora according to claim 1, wherein the agent is used for increasing the numbers of bacteria of genus Bifidobacterium and bacteria of genus Lactobacillus in the intestine.

6. A pharmaceutical preparation for oral administration for improving intestinal flora, comprising the agent for improving intestinal flora according to claim 1.

7. A food additive for improving intestinal flora, comprising the agent for improving intestinal flora according to claim 1.

8. A food or drink for improving intestinal flora, comprising the agent for improving intestinal flora according to claim 1.

9. A method for improving intestinal flora comprising orally applying (i) a lipase derived from a koji mold and/or (ii) a cellulase to an individual in need of improvement of the intestinal flora.

10. A method of manufacturing an agent for improving intestinal flora comprising: using (i) a lipase derived from a koji mold and/or (ii) a cellulase.

11. (canceled)


SEQUENCE LISTING (4 sequences)

SEQ ID NO: 1 - DNA, 17 bp, Artificial Sequence
  synthetic construct
cgcgtcyggtgtgaaag

SEQ ID NO: 2 - DNA, 18 bp, Artificial Sequence
  synthetic construct
ccccacatccagcatcca

SEQ ID NO: 3 - DNA, 22 bp, Artificial Sequence
  synthetic construct
gaggcagcagtagggaatcttc

SEQ ID NO: 4 - DNA, 27 bp, Artificial Sequence
  synthetic construct
ggccagttactacctctatccttcttc